(19) United States
(12) Patent Application Publication
(10) Pub. No.: US 2022/0346658 A1
(43) Pub. Date: Nov. 3, 2022

(54) WIRELESS IMPLANTABLE PASSIVE PRESSURE SENSOR

(71) Applicant: Purdue Research Foundation, West Lafayette, IN (US)

(72) Inventor: Rahim Rahimi, West Lafayette, IN (US)

(73) Assignee: Purdue Research Foundation, West Lafayette, IN (US)

(21) Appl. No.: 17/733,469

(22) Filed: Apr. 29, 2022

Related U.S. Application Data
(60) Provisional application No. 63/182,311, filed on Apr. 30, 2021.

(51) Int. Cl. A61B 5/03; A61B 5/00

(52) U.S. Cl. CPC A61B 5/036; A61B 5/6847; A61B 2562/0247; A61B 2562/12

(57) ABSTRACT

A microfluidic pressure sensor may include a bioinert shell having a cavity disposed therein. The cavity may include a reservoir and hydrophobic channel fluidly connected to the reservoir. Changes in pressure outside of the microfluidic pressure sensor may cause at least a portion of the shell to inflect into the reservoir thereby the fluid to move into the channel. The microfluidic pressure sensor may be bodily injected and the fluid level in the cavity may be detected using an ultrasound. The fluid level may be translated into a pressure measurement. The microfluid pressure sensor and uses thereof are suitable for bodily pressure measurements, including intra-abdominal pressure.

### CROSS REFERENCE TO RELATED APPLICATIONS

This application claims the benefit of U.S. Provisional Application No. 63/182,311 filed Apr. 30, 2021, the entirety of which is herein incorporated by reference.

### TECHNICAL FIELD

This disclosure relates to microfluidic pressure sensors and, in particular, to microfluidic pressure sensors and ultrasonic sensing.

### BACKGROUND

Intra-abdominal hypertension (IAH) is a condition defined by the World Congress of Abdominal Compartment Syndrome (WCACS), as the abnormally increased pressure in the abdomen. Based on pressure levels, IAH can be classified into 4 different grades: Grade I, II, Ill, and IV. Patients that are in Grade III or IV IAH (intra-abdominal pressure >2.7kPa), are at high risk for developing either acute or subacute Abdominal Compartment Syndrome (ACS). The increased pressure reduces blood flow to abdominal organs and will result in multiorgan dysfunction including pulmonary, cardiovascular, renal, portosystemic, and central nervous system. Delay in diagnosis or treatment of ACS has been shown to result in a mortality rate of 80% to 100%.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1A-B illustrate an example of a passive microfluidic pressure sensor.

FIG. 2 illustrates an exploded view of an example of a microfluidic pressure sensor.

FIG. 3 illustrates a perspective view of a microfluidic pressure sensor being filled with a fluid.

FIG. 4 illustrates an example of a microfluidic pressure sensor exposed to various external various pressures.

FIG. 5 illustrates on example of a measurement system for the microfluidic pressure sensor.

FIG. 6 illustrates an example of flow diagram for measuring bodily pressure using a microfluidic pressure sensor.

FIG. 7 illustrates a flow diagram for manufacturing a microfluidic pressure sensor.

### DETAILED DESCRIPTION

Accurate and reliable monitoring of intra-abdominal pressure is crucial for reduction of morbidity and improvement of overall survival and wellbeing in patients with high risk of Intra-abdominal hypertension (IAH) and/or Abdominal Compartment Syndrome (ACS). As World Congress of Abdominal Compartment Syndrome (WCACS) recommends, standard procedure for monitoring ACS and IAH is to measure intra-abdominal pressure (IAP) every 4-6 hours or per hour once evolving organ dysfunction until conditions have improved. One of the gold standard methods in clinical practice today for monitoring of IAP is the indirect intravesicular technique. This method was originally introduced in the 1980′s by Kron et al., which consisted of insertion of a balloon-tipped catheter into intra-abdominal organs (e.g., stomach or bladder) that transfers the IAP through their walls. The catheter is filled with a non-compressible fluid and connected to a water manometer or pressure transducer to obtain the pressure within the organ. Despite the simplicity and relatively non-invasiveness of this technique, it has inherent problems and measurement inaccuracy due to several limitations such as placing of the sensors at a relatively far distance from the pressure source, high risks of having air bubbles in the line, sensitive to body position, poor dynamic response with damping caused by the elastic tubing and connections. Although newer intravesical pressure (IVP) monitoring methods have tried to address these measurement inaccuracies by using sensor-tipped catheters in conjunction with different wired or wireless readout electronics, they are not always a reliable and direct representation of IAP. Furthermore, IVP depends on the physiological function of the bladder which can be unreliable or unfeasible in patients with bladder trauma, outflow obstruction, or pelvic hematomas. IAP can also be more accurately measured by directly placing sensor-tipped catheters into the peritoneal cavity. However, currently available catheter based methods for directly measuring IAP are highly invasive with high risk of infection by having an exposed wound at the site of catheter insertion in the body [4].

Therefore, a direct measurement technique that is capable of providing an in-situ wireless monitoring of IAP, is preferred. One such way is via a miniaturized implantable telemetry system that monitors the IAP and wirelessly transmits the intra-abdominal pressure levels to a computer. In general, such systems can be designed in both active and passive wireless data transmission scheme. Active systems are often designed with an internal power source that provides the energy for operating the onboard electronics for performing measurement and transmitting the readings to a nearby receiver. However, internal batteries used in such devices will need to be recharged or replaced and oftentimes contribute to a larger implant size. In contrast, passive wireless pressure sensing schemes use inductive coupling to power and interrogate the pressure readings from the implanted device. A typical example of such devices is to use a MEMS (Micro electromechanical system) based passive LC (inductive or capacitive) oscillator, in which either the coil or the capacitor is pressure sensitive. Although passive telemetry systems have a significantly smaller footprint and increased lifetime of operation, they frequently have complicated fabrication process and require complex and costly readout equipment (e.g., network analyzer). Furthermore, these systems are highly inefficiency in powering deeply implanted and small size (mm-scale) devices due to energy dissipation that occurs in conductive tissue as well as the small size of the receiver coil on the device. In contrast, ultrasonic wave has a smaller wavelength with less attenuation in biological tissues, providing a more efficient way of energy transmission and ability to couple with smaller implanted devices.

Using this unique property, several groups have demonstrated different implantable devices that utilize piezoelectric transducer as an energy harvesting unit to convert ultrasonic wave into electrical energy for powering on board sensors and electronics for different sensing and stimulating applications. Despite the small mm-scale size of such implantable devices, they suffer from a major drawback of the need for converting the ultrasonic signal to RF signal for data transmission, which increases the complexity of the readout system outside the body by requiring both an ultrasonic powering and RF reader for each measurement. Furthermore, there is a concern of lead toxicity of such devices due to the high levels of lead and its compounds present in the piezoelectric elements (e.g., PZT (lead zirconate titanate)). Over the past several decades, ultrasound imaging technologies have continued to improve in terms of both image quality and miniaturization at a rapid pace. More recent examples are handheld imaging systems that have created new opportunities in rapid and nondestructive point-of-care diagnostics such as detecting different tissue conditions inside the body.

Nevertheless, there is still uncertainty in its ability to be used for direct assessment of hydrostatic pressure inside the tissue and it often needs to be used in an indirect approach of imaging of gas filled implanted objects which will change in ultrasound reflectivity with applied pressure. These indirect methods can be categorized into two main groups of microbubble and fluidic-based systems. In the microbubble-based approach, the pressure sensitive microbubble is simply introduced into the targeted tissue. However, they have low sensitivity to small pressure changes and are hard to maintain at a certain location for continuous tracking of pressure changes. Fluidic-based approaches provide a higher sensitivity with repeatable long-term performance by implanting a barometric device, in which the liquid level inside the reservoir is indicative of the measured parameter (e.g., a pressure or strain). For instance, Limbrick developed an intracranial pressure monitor system, which uses a subdural fluid bladder and connecting catheter that upon applied pressure forces the fluid into a registered multichannel to monitor the position of an air-fluid interface. Although the design enabled ultrasound imaging as the interrogation approach, the device requires assembly of multiple components (consisting of a subdural fluid bladder, multichannel indicator, and a catheter connecting these components) which increases the overall size of the final device (>10cm) and also adds to the manufacturing complexity and possibility of failure at some points throughout the device and leakage of the internal fluid. Furthermore, the large size of the device necessitates large incision (>3cm) for implantation and limits its use to monitoring chronic conditions (e.g. intracranial pressure monitoring) which makes it challenge to be adapted for noninvasive implants with short-term applications especially for measurement of intra-abdominal pressure (e.g. less than 7days).

Accordingly, there is disclosed a microfluidic pressure sensor device and related methods for internal bodily pressure sensing via ultrasonic imaging. The device may be monolithically microfabricated with soft and biocompatible material in a form factor suitable for minimally invasive implantation into the abdominal cavity while also providing clear visibility with ultrasound imaging for remote interrogation of pressure levels.

By way of an introductory example, a microfluidic pressure sensor may include a first biocompatible layer, a second biocompatible layer thicker than the first layer; and an intermediate biocompatible layer defining a cavity. The first layer may cover at least a portion of the cavity on a first side of the device. The second biocompatible layer may cover another portion of the cavity on a second side of the device. Changes in pressure outside of the microfluidic pressure sensor cause the first layer to inflect into the cavity causing fluid to move along the cavity. The change in fluid location may be measured by an ultrasonic imaging device. Based on the fluid location, the pressure outside of the microfluidic pressure sensor may be calculated.

In some examples, the microfluidic pressure sensor be formed of a biocompatible material, such as polydimethylsiloxane (PDMS), and include hydrophobic micro-channel connected to a sub mm scale reservoir filled with water, which is well suited for wireless and passive pressure and force monitoring applications at various locations throughout the body. Specifically, the pressure sensing microfluidic system may include a sub mm scale reservoir connected to micro-channel, where the reservoir is filled with water and covered by a thin and pressure sensitive PDMS membrane. Increase in external pressure (e.g., high IAP) leads to flexure of the pressure sensitive membrane and pushes the water from the reservoir into the micro channel. The level of in channel fluid displacement directly corresponds to the elevated pressure of the environment, which can wirelessly be quantified by using simple ultrasound imaging.

In general, ultrasound imaging is obtained by the dissimilarity in acoustic impedances of two media, which determines the reflection degree of the propagating ultrasound wave; the higher reflection coefficient, the better ultrasound visualization. Water has a similar acoustic impedance to biological tissues, resulting in diminished contrast in the ultrasound image; whereas, air has a different acoustic impedance to tissues, generating a more enhanced contrast in the ultrasound image. In such way, the ultrasound imaging approach enables generation of a high-resolution visualization of the water within the reservoir against the air inside the channel, and thus allowing direct and quantitative pressure reading from the implanted microfluidic pressure sensor. The entire fabrication process of the sensor is fully compatible with industrial-scale micro-manufacturing, which permits scalable production at a significantly low unit-cost. Moreover, the device only requires a minimally invasive surgical procedure (e.g., biopsy needle) for implantation of the device to monitor IAP in-situ at an easy-to-operation, high-throughput, and tunable timely manner in patients suspected for IAH. Moreover, the device can be implanted by a minimally invasive surgical procedure (e.g., biopsy needle). These procedures usually take less than 10 to 15 minutes to perform under local anesthesia and require small skin incision, reducing the complications of insertion inside the body and allowing easy follow up readings with non-invasive ultrasonic imaging. In general, the demonstrated system has several advantages compared to catheters and other surgically implantable pressure sensors in terms of less complexity of operation and implantation in practice. Nevertheless, any form of cuts or needle insertion into issue is not immune to infection. Yet, this risk can be minimized by autoclaving the device before implantation and proper care of the site of implantation and using proper antiseptic procedure. Additional benefits, efficiencies, and improvements over existing solutions are made evident in the systems and methods described below.

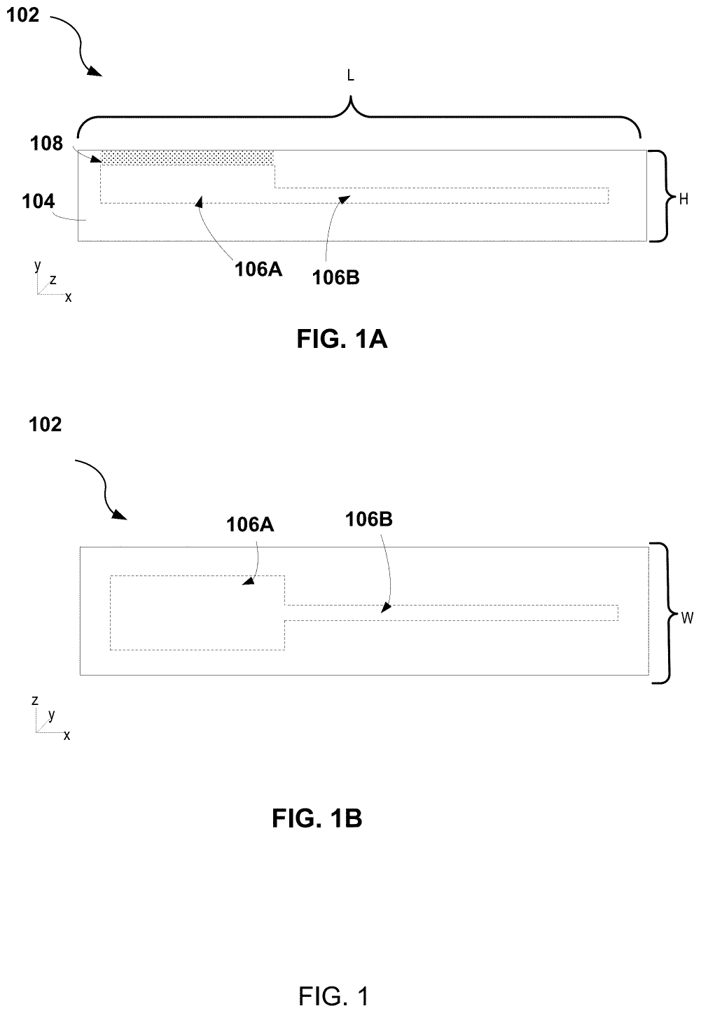

FIG. 1A-B illustrate an example of a passive microfluidic pressure sensor. The sensor may include shell 104 and a cavity 106A-B disposed inside of the shell 104.

The shell 104 may include a biocompatible material. In some examples, the biocompatible may include a silicon-based organic polymer, such as polydimethylsiloxane (PDMS), or some organic polymer. The shell may define the cavity 106A-B. The cavity may include an internal void that is at least partially filled with fluid. Changes in pressure outside of the microfluidic pressure sensor may cause shell 104 to inflect or flex into the cavity, or a portion thereof, displacing the fluid within the cavity in a manner that can be measured.

The cavity 106A-B may include a reservoir 106A and a microfluidic shell 106B. The reservoir 106A may include sub-millimeter scale tank that holds a fluid, such as water or some other fluid well suited for wireless and passive pressure and force monitoring applications at various locations throughout the body. The microchannel 106B may be fluidly connected to the 106A and extend from the reservoir 106A. For example, the microchannel 106B may be located immediately adjacent to and directly connected to the reservoir 106A such that the reservoir 106A and microchannel 106B are contiguous portions of the cavity 106A-B.

The microchannel 106B may be defined by hydrophobic material such as a flexible polymer (e.g. silicone). The hydrophobic material may cause the fluid to go back into the reservoir after the pressure is removed due to capillary action. In short this allows the repeatable performance of the sensor.

The shell may include a pressure sensitive zone 108. The pressure sensitive zone 108 may at least partially define a wall of the reservoir 106A and an outer surface of the microfluidic pressure sensor. As illustrated by the hatching in FIG. 1A, the pressure sensitive zone 108 is a wall of the reservoir 108A which is thinner than the other walls of the reservoir. Other configurations are possible depending on the size of the reservoir and the pressure sensitive zone may include other wall(s) that define the reservoir 108A.

In response to changes in pressure outside of the microfluidic pressure sensor 102, the shell 104 may inflect into the reservoir. The reservoir wall(s), or the portions thereof, may be large enough such that the shell 104 may inflect into the reservoir 106A. The microchannel wall(s) may be small enough such that the shell 104 does not inflect into the microchannel 106B. Furthermore, the microchannel dimensions can be modified in order to provide a wider range of pressure sensitivity.

The size and shape of the reservoir may provide a volume that can be adapted based on the application. In some examples, the volume may be around 25-300 cubic micrometers. For example, in various experimentation, the reservoir had an overall size of 1.6 mm x 1.6 mm x 0.8 mm (length x width x height) and the micro-channel is 8 mm in length, 0.45 mm in width, and 0.45 mm in height. The volume of reservoir is designed to be slightly larger than that of micro-channel in order to provide enough water to fill the entire channel when encountering high pressure outside of the device.

While the overall dimensions of the microfluidic pressure sensor may vary depending location of application in the body, a small form factor is an advancement of the device and methods described herein. By way of example, the height (H) of the microfluidic pressure sensor may be 25 μm or less, the length (L) of the microfluidic pressure sensor may be or less, and the width (VV) of the pressure sensor may be 25 μm, or less.

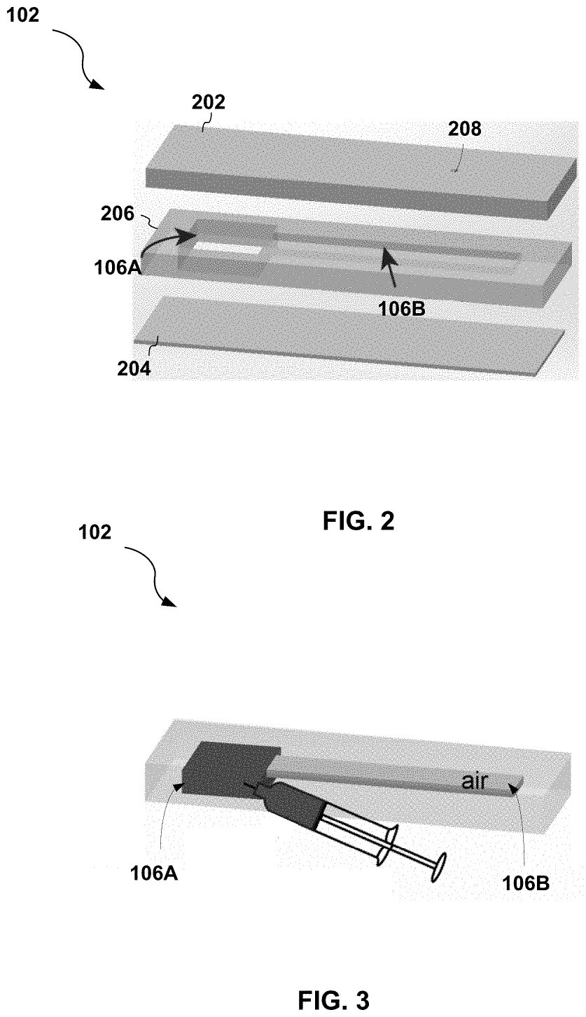

FIG. 2 illustrates an exploded view of an example of the microfluidic pressure sensor 102. The microfluidic pressure sensor 102 may include a thick polymer layer 202, a thin polymer layer 204, and an intermediate polymer layer 206.

The intermediate layer 206 may define the cavity 106A-B. Thus, the cavity 106A-B, or a portion thereof, may extend from a first side of the intermediate layer 206 to the second side of the intermediate layer. The thin layer 204 may at least partially cover the cavity. The thick layer 202 may cover the cavity 106A-B on the second side of the sensor 102.

The thick PDMS layer may be able to resist mechanical deflection at high pressures. The thin layer 204 may provide a pressure-sensitive membrane for the reservoir 106B. For example, the thin layer 204 may form a pressure sensitive wall of the reservoir 106B. The thin layer 202 may be thinner than the thick layer 204. For example, the thin layer 202 may have a thickness between 10 micrometers to 100 micrometers. The thick layer 204 may have a thickness between 500 micrometers to 2 mm. Alternatively or in addition, the thin layer 202 may have a thickness such that flexure occurs at a pressure below 12 KPA . Under the same pressure, the thick layer may not flex into or out of the reservoir (or such flexure may be minimal). Accordingly, the thin layer 204 may provide the pressure sensitive zone for the reservoir 106B. It should be appreciated, however, that other variations are possible. For example, the thick layer may instead be another thin layer forming additional pressure sensitive zones. Also, it should be noted that the area of the walls of the reservoir may influence the thicknesses of the layers to achieve pressure-sensitivity and rigidity.

In some examples, the micro-channel may shorter than the reservoir. The additional material below the micro-channel may also serves as a high-pressure-resistant component to eliminate its shape deformation with applied pressure.

In some examples, the microfluidic pressure sensor 102 may include an aperture 208. The aperture 208 may include a hole through one or more of the layers that extends into the microchannel. The aperture 208 may allow the fluid to transfer into microfluidic channel with the application of pressure to the reservoir. The aperture may be sealed up after the fluid is injected. In some cases, the microfluidic pressure sensor 102 may be made with a self-sealing polymer, such as PDMS.

FIG. 3 illustrates a perspective view of the microfluidic pressure sensor 102 being filled with a fluid. A solution may be injected into the reservoir via syringe or other suitable injection method. The solution may include a fluid, such as water, which is well suited for wireless and passive pressure and force monitoring applications at various locations throughout the body. In some examples, the solution may be colored or include other properties which make it easy to recognize using ultrasound imaging.

After injecting the microfluidic pressure sensor 102 with the solution, the reservoir 106A may receive the solution. A pressure sensitive deflectable membrane may push the fluid from the reservoir 106A into micro-channel 106B. Increases in pressure outside of the microfluidic pressure sensor 102 relative to inside the cavity means that the fluid will flow further along the microchannel 106B as the air or gas portion in the microchannel 1066 compresses. Conversely, decreasing pressure outside the microfluidic pressure sensor 102 relative to inside the cavity will result in less fluid occupying the microchannel 106B. It should be noted that changing the thickness of the thin layer 204 (FIG. 2) will allow easy adjustment of the sensitivity and working range of the pressure sensors.

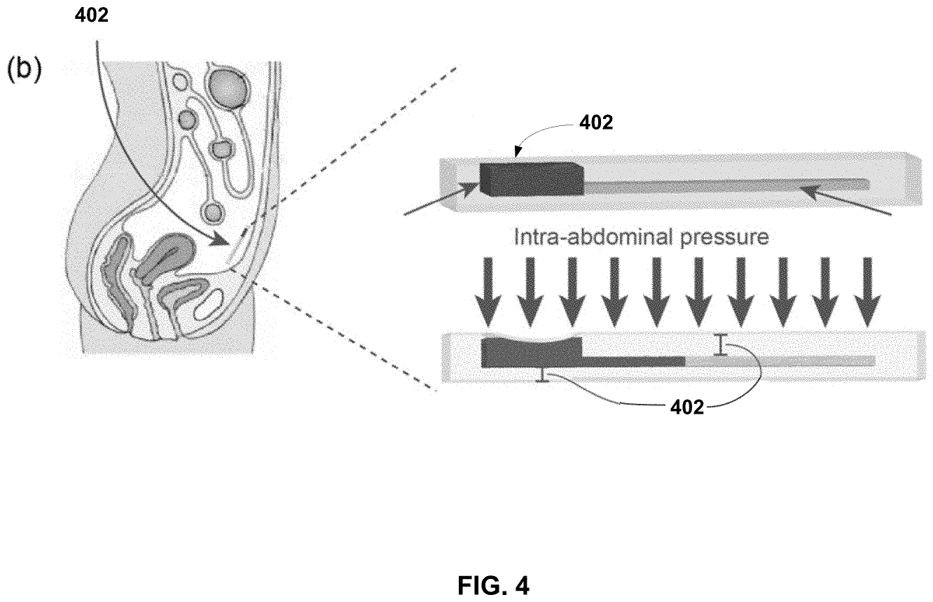

FIG. 4 illustrates an example of the microfluidic pressure sensor at various pressures. The hydrophobic micro-channel may be connected to the sub millimeter scale reservoir and filled with a fluid, such as water, which is well suited for wireless and passive pressure and force monitoring applications at various locations throughout the body. The reservoir may be covered by a thin and pressure sensitive PDMS membrane layer ,which creates the pressure sensitive zone 108. Increase in external pressure (e.g., high IAP) leads to inflection of the pressure sensitive membrane and pushes the water from the reservoir into the micro channel. The level of in channel fluid displacement directly corresponds to the elevated pressure of the environment, which can wirelessly be quantified by using simple ultrasound imaging. Pressure insensitive zones 402 may resist inflection into portion of the reservoir and/or channel.

The microfluidic pressure sensor suitable for placement in various locations in the body. As illustrated in FIG. 4 and other examples described herein, the sensor may be placed in the abdominal activity 402. However, other locations are possible, including, for example, periphery hands and legs to measure compartment syndrome.

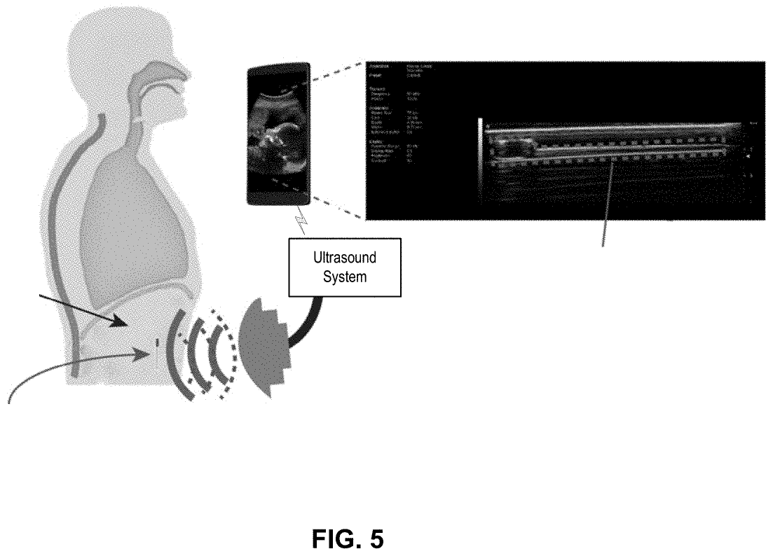

FIG. 5 illustrates on example of a measurement system for the microfluidic pressure sensor. The system may include an ultrasound system. Ultrasound imaging is obtained by the dissimilarity in acoustic impedances of two media, which determines the reflection degree of the propagating ultrasound wave; the higher reflection coefficient, the better ultrasound visualization. Water has a similar acoustic impedance to biological tissues, resulting in diminished contrast in the ultrasound image; whereas, air has a different acoustic impedance to tissues, generating a more enhanced contrast in the ultrasound image. In such way,

Accordingly, an ultrasound transducer of the ultrasound system may be directed to the abdominal cavity, or other locations of the body where the microfluidic pressure sensor is located. The ultrasound system generate a high-resolution visualization of the water within the reservoir against the air inside the channel. The image may be displayed on a device, thus allowing direct and quantitative pressure reading from the implanted microfluidic pressure sensor.

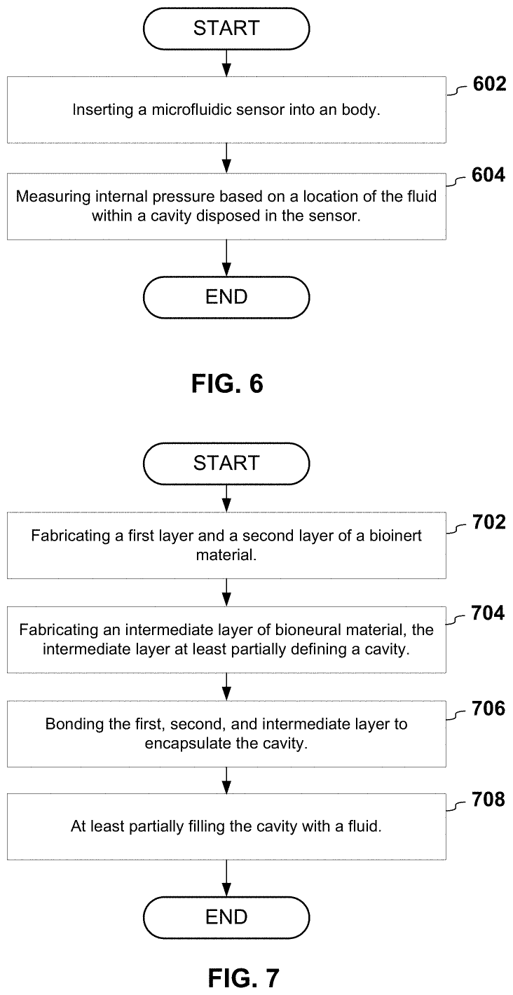

FIG. 6 illustrates an example of flow diagram for measuring bodily pressure using the microfluidic pressure sensor. The microfluidic pressure sensor may be inserted into the abdominal cavity, or some other location on the body (602). The injection may occur thought biopsy implantation process.

Bodily pressure may be measured based on a location of the fluid within the internal cavity of the sensor (604). To measure the intrabdominal pressure, an ultrasound transducer may be directed at a location of the body having the microfluidic pressure sensor. The ultrasound imaging data may be displayed. A physician or medical professional may record the location or level of fluid within the microfluidic channel. The physician may convert the level measurement

A calibration curve may be established by measuring the liquid level in the microchannel for various pressures applied to the pressure sensitive zone of the reservoir. The calibration curve may be used to establish a correlation function to map liquid level measurements to external pressure readings. The correlation function may be used for to make measurements with sensors that are similar or identical to the sensor used to establish the calibration curve. The calibration process can be performed once for a new sensor design and be applied for all the fabricated sensors. Calibration processes may be performed using an ultrasonic imaging.

FIG. 7 illustrates a flow diagram for manufacturing the microfluidic pressure sensor. The microfluidic pressure sensor may be manufactured through a layer-by-layer scalable assembly process via plasma bonding of three layers. The first and second layer may be fabricated (702). The first and second layers may have the with the appropriate thicknesses to establish the pressure sensitivity for the walls of the reservoir. In some examples, the first layer may be thinner than the second layer. Thus, the first layer may form a pressure sensitive wall. The second layer may be pressure insensitive and able to resist mechanical deflection at high pressures.

The intermediate layer may also be fabricated and define the cavity of the sensor (704). The cavity can be created through simple laser cutting of the silicone material or using standard micro machining and soft lithography processes

The first, second, and intermediate layers may be bonded to encapsulate the cavity (706). The bonding may occur through, for example, standard plasma surface treatment and heat press, though other techniques are possible.

Finally, the reservoir may be at least partially filled with liquid (708). In various extermination, approximately 2 μl of water using a 30-gauge hypodermic needle was used to inject the water into the reservoir. During the water injection, a 0.25 mm diameter aperture at end of the channel is kept open for air ventilation and later sealed with silicone glue after filling the water to the entire reservoir. This procedure increases the ease of filling the reservoir and further enhances the structural robustness of the device by balancing the internal air pressure to the surrounding environment before operation.

The microfluidic pressure sensor and system described herein may be implemented with additional, different, or fewer components than illustrated. Each component may include additional, different, or fewer components. Moreover, the steps of any method described herein may include additional and/or fewer steps. The steps may be implemented in other orders.

While various embodiments have been described, it will be apparent to those of ordinary skill in the art that many more embodiments and implementations are possible. Accordingly, the embodiments described herein are examples, not the only possible embodiments and implementations.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

What is claimed is:

1. A microfluidic pressure sensor comprising:
a bioinert shell having a cavity disposed therein, the cavity comprising a reservoir and hydrophobic channel fluidly connected to the reservoir,
wherein changes in pressure outside of the microfluidic pressure sensor cause at least a portion of the shell to inflect thereby causing the reservoir to deform and the fluid to move into the channel.

2. The microfluidic pressure sensor of claim 1, wherein the at least the portion of the shell inflects into the reservoir an external pressure of 12 KPA or lower.

3. The microfluid device of claim 1, wherein the bioinert shell comprises a first layer, a second layer, and an intermediate layer, wherein the intermediate layer is positioned in between the first layer and second layer, wherein the first layer at least partially defines a pressure-sensitive wall of the reservoir which inflects into the reservoir in response to increased pressure outside of the reservoir.

4. The microfluidic pressure sensor of 3, wherein the first layer at least partially defines the reservoir and the channel, and the second layer at least partially defines the reservoir but not the channel.

5. The microfluidic pressure sensor of claim 1, further comprising an aperture defined in shell at an end of the channel opposite the reservoir.

6. The microfluidic pressure sensor of claim 1, wherein the fluid is colored.

7. The microfluidic pressure sensor of claim 1, wherein the shell comprises Polydimethylsiloxane (PDMS).

8. The microfluidic pressure sensor of claim 1, wherein a maximum length of the microfluidic pressure sensor does not exceed 55 kPa a maximum height does not exceed 15 mm and a maximum width does not exceed 4mm

9. A method, comprising:
inserting a microfluidic pressure sensor into a body, the microfluidic pressure sensor comprising a fluid predisposed in an internal cavity of the microfluidic pressure sensor; and
measuring bodily pressure based on a location of the fluid within the internal cavity.

10. The method of claim 9, wherein cavity comprises a reservoir and a channel immediately adjacent to the reservoir.

11. The method of claim 10, wherein the reservoir is at least partially filled with the fluid.

12. The method of claim 9, further comprising:
directing an ultrasound probe at the body;
displaying, based on the image data, the microfluidic pressure sensor in the abdominal cavity; and
identifying the location of the fluid within the internal cavity of the sensor.

13. The method of claim 9, where measuring internal pressure based on a location of the fluid within the channel further comprises:
converting a measured location of the fluid in the internal cavity of the sensor to a pressure sensor based on a correlation function that characterizes previously measured pressures and fluid levels.

14. The method of claim 9, wherein the inserting a microfluidic pressure sensor into a body comprises inserting the microfluidic sensor into an abdominal cavity, wherein measuring bodily pressure comprises measuring intra-abdominal pressure.

15. A method of manufacturing a microfluidic pressure sensor comprising:
forming a base polymer layer, forming an intermediate polymer layer that defines a reservoir and a microchannel, the reservoir being open on a first side of the intermediate polymer layer and the microchannel being open on a second side of the intermediate polymer layer;
forming a top polymer layer that is thinner than the base polymer layer;
bonding the top polymer layer to the intermediate layer to cover the reservoir and form a pressure-sensitive wall of the reservoir; and
bonding the base polymer layer to the intermediate layer to enclose the channel.

16. The method of claim 11, further comprising:
injecting a fluid into the reservoir;

17. The method of claim 12, further comprising:
varying the pressure outside of the microfluidic pressure sensor to cause the outer layer to flex into the cavity; and
characterizing the location of the fluid in the microchannel a plurality of controlled pressure values.

18. The method of claim 15, wherein base polymer layer has a thickness between 10 micrometers to 100 micrometers.

19. The method of claim 15, wherein the base layer, the intermediate polymer layer, and the top polymer layer each comprise Polydimethylsiloxane (PDMS).
